(19) United States
(12) Patent Application Publication
(10) Pub. No.: US 2014/0073594 A1
(43) Pub. Date: Mar. 13, 2014

(54) GLYCOMIMETIC COMPOUNDS AND METHODS TO INHIBIT INFECTION BY HIV

(71) Applicant: GlycoMimetics, Inc., Gaithersburg, MD (US)

(72) Inventors: John L. Magnani, Gaithersburg, MD (US); Arun K. Sarkar, North Potomac, MD (US)

(73) Assignee: GlycoMimetics, Inc., Gaithersburg, MD (US)

(21) Appl. No.: 14/080,926

(22) Filed: Nov. 15, 2013

Related U.S. Application Data
(63) Continuation of application No. 13/081,068, filed on Apr. 6, 2011, now abandoned.
(60) Provisional application No. 61/321,740, filed on Apr. 7, 2010.

(51) Int. Cl. C07H 15/207

(52) U.S. Cl. CPC C07H 15/207
USPC 514/35; 536/17.9

(57) ABSTRACT

Compounds, compositions and methods are provided for use to inhibit infection by human immunodeficiency virus (HIV). More specifically, the present invention relates to glycomimetic compounds that inhibit HIV infection, and uses thereof.

### CROSS REFERENCE TO RELATED APPLICATION

This application claims the benefit under 35 U.S.C. §119(c) of U.S. Provisional Patent Application No. 61/321,740 filed Apr. 7, 2010, which application is incorporated herein by reference in its entirety.

### BACKGROUND

1. Technical Field

The present invention relates generally to compounds, compositions and methods for prevention or treatment of HIV infection. More specifically, the present invention relates to glycomimetic compounds that inhibit HIV infection, and uses thereof.

2. Description of the Related Art

Acquired Immune Deficiency Syndrome (“AIDS”), a fatal human disease, is generally considered to be one of the more significant diseases to affect humankind, and has affected numerous individuals worldwide. The disease appears to have originated in Africa and then spread to other locations, such as Europe, Haiti and the United States. AIDS began to be recognized as a distinct new disease in about the mid-1970s.

Due to the devastating effect of AIDS on patients and indications that the disease was spreading, much effort has been devoted to elucidate and identify the cause of the disease. Epidemiological data suggested that AIDS is caused by an infectious agent that is transmitted by exposure to blood or blood products. Groups reported to be at greatest risk of contracting AIDS include homosexual or bisexual males and intravenous drug users. Hemophiliacs who receive blood products pooled from donors and recipients of multiple blood transfusions are also at risk.

AIDS is a disease that damages the body's immune system, leaving victims susceptible to opportunistic infections, malignancies or other pathological conditions against which a normal immune system would have protected the subject. Clinical manifestations of the disease in its final stage include a collapse of a patient's immune defenses (which generally involves a depletion of helper T cells) accompanied by the appearance of a Kaposi sarcoma and/or various opportunistic infections. The pronounced depression of cellular immunity that occurs in patients with AIDS and the quantitative modifications of subpopulations of their T lymphocytes suggests that T cells or a subset of T cells are a central target for the infectious agent.

The etiology of AIDS and related disorders has been identified as being associated with infection by a class of lymphotrophic retrovirus termed human immunodeficiency virus (HIV; known previously as HTLV or LAV). The virus is spread when body fluids, such as semen, vaginal fluids or blood, from an infected individual are passed to an uninfected person. As noted above, AIDS is characterized by a disorder associated with an impaired cell-mediated immunity and lymphopenia, in particular, depletion of those T ceils that express the T4 (CD4) glycoprotein. This is due to the fact that HIV preferentially infects the CD4 lymphocyte population (CD4 cells). Both the binding of virus to susceptible target cells and the cell fusion that is a characteristic manifestation of HIV-induced cytopathology involve specific interactions between glycoproteins in the viral envelope and the cell surface of CD4 cells.

HIV contains two heavily glycosylated external envelope proteins. gp120and gp41, which mediate attachment of virions to glycosylated cell surface receptor molecules. These glycoproteins are encoded by the env gene and translated as a precursor, gp160, which is subsequently cleaved into gp120 and gp41. Gp120 binds to the CD4protein present on the surface of helper T lymphocytes, macrophages, and other cells, thus determining the tissue selectivity of viral infection.

The CD4 protein is a glycoprotein of approximately 60,000 molecular weight and is expressed on the cell membrane of mature, thymus-derived (T) lymphocytes, and to a lesser extent on cells of the monocyte-macrophage lineage. CD4 cells appear normally to function by providing an activating signal to B cells, by inducing T lymphocytes bearing the reciprocal CD8 marker to become cytotoxic/suppressor cells. and/or by interacting with targets bearing major histocompatibility complex (MHC) class II molecules. The CD4 glycoprotein in addition to playing an important role in mediating cellular immunity also serves as the receptor for HIV.

Other HIV has infected a cell, it replicates to increase the number of copies of the virus. Replication of the HIV genome proceeds by a series of enzymatic reactions involving two virus-encoded enzymes, reverse transcriptase (“HIV RT”) and integrase, as well as host cell-encoded DNA polymerases and RNA polymerase. HIV RT polymerizes deoxyribonucleotides by using viral RNA as a template and also acts as a DNA polymerase by using the newly synthesized minus strand DNA as a template to produce a double-stranded DNA. More specifically, HIV RT copies the viral RNA to yield an RNA-DNA hybrid. The RNA strand of the hybrid is degraded and then the viral polymerase copies the resultant single-stranded DNA to yield a double-stranded DNA. The latter is integrated into the host cell genome.

Due to the essential role of HIV RT in the invasion of a host organism by the virus, therapeutic approaches have been based upon an attempt to inhibit HIV RT or to incorporate nucleoside analogs that terminate viral DNA synthesis. The most commonly-used drugs against HIV RT are chain terminators. These molecules are presumably incorporated into the polynucleotide chain by HIV RT, but are unable to be extended on subsequent nucleotide additional steps. For example, azidothymidine (“AZT”), one of the most commonly used drugs for the treatment of AIDS, is directed against HIV RT. However, even these inhibitors of HIV RT have been limited in success because of the extensive genetic variation and high mutation rate of HIV. Therefore, by rapid evolution of HIV, mutations in HIV RT arise frequently in infected individuals and render the virus resistant to HIV RT inhibitors. This is a significant drawback to conventional therapies.

Although a few drugs such as AZT have prolonged the lives of some individuals with AIDS, there is presently no cure for AIDS. Therapeutic agents are needed for all stages of AIDS infectious. Due to the limited success for previously suggested compositions tor the treatment of AIDs, there is a need in the art for new therapies. The present invention fills this need, and further provides other related advantages. BRIEF SUMMARY

Briefly stated, compounds, compositions and methods tor preventing or treating HIV infection are provided.

The present invention in an embodiment provides a compound for inhibiting HIV infection, where the compound consists of a naphthalene, a phenalene, an anthracene, a phenanthrene or an acenaphthylene, joined to at least two glycomimetics selected independency from glycomimetics having the formula:

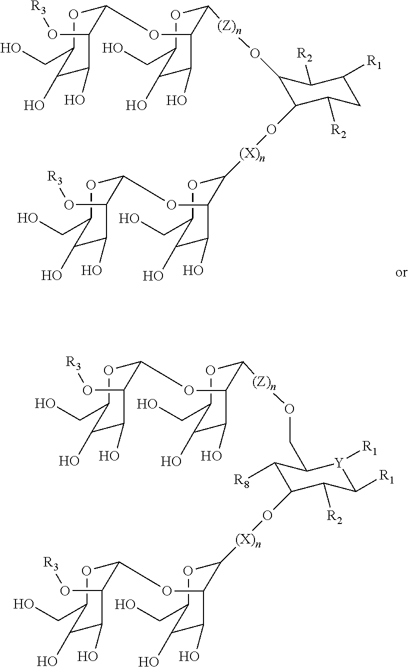

wherein:

n=independently selected from 0-1;

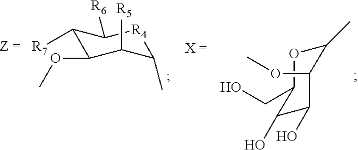

Y=C or O;

R₁=independently selected from H, C(═0)OCH, L, with the provisos where there are two Rᵢ on the same glycomimetic that both Rᵢ are not H or U and where Y is O that there us no R₁ at Y;

R₂ =independently selected from H, C₁-C₈ alkanyl, C₂-C₈ alkenyl, C₂-C₈ alkynyL halogenated C₁-C₈ alkanyl, aryl or heterocycle either of which may be substituted with one or more of Me, OMe, halide, OH, or NHX where X=H, C₁-C₈ alkanyl, C₂-C₈ alkenyl, C₂-C₈ alkynyl, halogenated C₁-C₈ alkanyl, aryl or heterocycle cither of which may be substituted with one or more of Me, OMe, halide, or OH; —C(═0)OX where X is C₁-C₈ alkanyl, C₂C₈ alkenyl, C₂-C₈ alkynyl, aryl or heterocycle either of which may be substituted with one or more of Me, OMe, halide, or OH; —C(═O)NH(CH₂)ₙNH₂ where n=0-30, C(═0)NHX or CX₂OH, where X=C₁-C₈ alkanyl, C₂-C₈ alkenyl, C₂-C₈alkynyL halogenated C₁-C₈ alkanyl, aryl or heterocycle either of which may be substituted with one or more of Me, OMc, halide, or OH; OC(═O)X, OX, NHX, NH(═0)X, where X=H, C₁-C₃ alkanyl, C₂-C₈ alkenyl, C₂-C₈ alkynyl, halogenated C₁-C₈ alkanyl, aryl or heterocycle either of which may be substituted with, one or more of Me, OMe, halide, or OH;

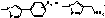

where R₉=F, NH₂, C₁-C₈ alkanyl, C₂-C₈ alkenyl, aryl, COOH, or COOR₁₀, R₁₀=C₁-C₈ alkanyl, C₂-C₈ alkenyl, or aryl, R₁₁=C₁-C₈ alkanyl, C₂-C₈ alkcnyl, or C(═O)R₁₂, R₁₂=C₁-C₈ alkanyl, C₂-C₈alkenyl, or aryl;
R₃=H, mannose;
R₅=H, C₁-C₈ alkanyl,aryl,

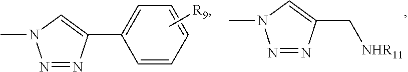

where R₉=F, NH₂, C₁-C₈ alkanyl, C₂-C₈ alkenyl, aryl, COOH, or COOR₁₀, R₁₀=C₁-C₈ alkanyl, C₂-C₈ alkenyl, or aryl, R₁₁=C₁-C₈ alkanyl, C₂-C₈ alkenyl, Or C( O)R₁₂, R₁₂=C₁-C₈ alkanyl, C₂-C₈ alkenyl, or aryl;
R₆=H, C₁-C₈ alkanyl, aryl, CH₂OH,

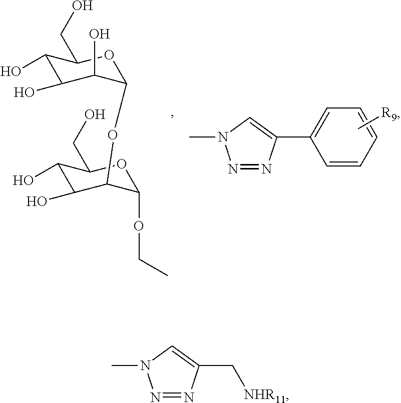

where R₉=F, NH₂, C₁-C₈ alkanyl, C₂-C₈ alkenyl aryl, COOH or COOR₁₀, R₁₀-32 C₁-C₈ alkanyl, C₂-C₈ alkenyl, or aryl, R₁₁=C₁-C₈ alkanyl, C₂-C₈ alkenyl, or C(═O)R₁₂, R₁₂=C₁-C₈ alkanyl, C₂-C₈ alkenyl, or aryl;

R₇=H,OH;

R₈ =H, OH, CH₃, —(CH₂)ₘCH₃ where m=1-20; and

L is a linker to which the glycomimetic is covalently joined to the naphthalene, phenalene, anthracene, phenanthrene, or acenaphthytene.

A compound of the present invention may be covalcntly joined (linked) to a vaccine carrier.

Compositions are formed by combining a compound of the present invention (with or without a vaccine carrier) with a pharmaceutically acceptable carrier or diluent.

The present invention provides a method for inhibiting HIV infection in an individual comprising administering to the individual in an amount effective to inhibit HIV infection a compound of the present invention, thereby inhibiting the HIV infection.

A compound or composition of the present invention can be used to develop therapeutic antibodies (e.g., monoclonal antibodies).

A compound or composition of the present invention can be used as an inhibitor of HIV infection or in the manufacture of a medicament, for example, for any of the uses recited herein.

These and other aspects of the present invention will become apparent upon reference to the following detailed description and attached drawings. All references disclosed herein are hereby incorporated by reference in their entirety as if each was incorporated individually. The chemical formulae set forth herein are depicted without regard to axial or equatorial forms or projections.

### BRIEF DESCRIPTION OF THE DRAWINGS

FIG. 1 (Fig. 1A, Fig. 1B and Fig. 1C) is a diagram illustrating the synthesis of a glycomimetic.

Figure 2 is a diagram illustrating the synthesis of a compound of the present invention,

### DETAILED DESCRIPTION

As noted above, the present invention provides compounds, compositions and methods for use in preventing (prophylaxis) or treating HIV infection. The compounds have a variety of uses in vitro and in vivo, including for use to inhibit HIV infection. A compound of the present invention may comprise, or consist of, the compounds disclosed herein, a portion of which, may include any of the formulae depicted herein. The compounds include, or consist of, a naphthalene, phenalene, anthracene, phenanthrene or acenaphthylene, to which is covaicntfy joined at least two (i.e, two or more up to ten including any whole integer in-between) glycomimetics. The glycomimetics are independently selected, i.e., the glycomimetics may be the same or different. Where there are more than two glycomimetics in a compound, it is possible to also have some, but not all, of the glycomimetics the same in the compound.

All compounds of the present invention or useful thereto (e.g., for pharmaceutical compositions or methods of preventing or treating) include physiologically acceptable salts thereof. Examples of such salts are Na, K, Li, Mg, Ca and CI.

In one embodiment, at least one of the glycomimetics of the compound has the formula:

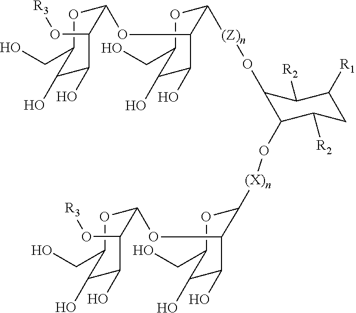

In another embodiment, at least one of the giyeomimetics of the compound has the formula:

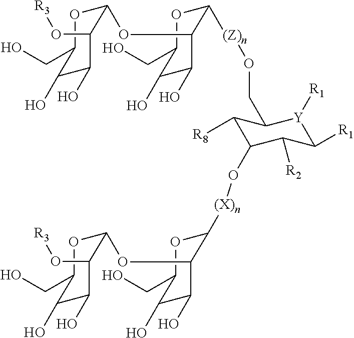

Y is either carbon or oxygen. In one embodiment, Y is carbon.

The glycomimctics of the above formulae may possess a variety of substiruents via the R. groups, and n (which may be 0 or 1) is independently selected for (X)ₙ and (Z)ₙ. Thus, each glycomimetic of the compounds may possess no X and Z; no X and one Z; one X and DO Z; or one X and one X.

Where n is 0 for (X)ₙ, there is no X present. Where n is 1 for (X)ₙ, X is present. X is

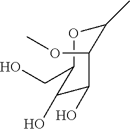

Where n is 0 for (Z)ₙ, there is no Z present and the glycomimetics of the compounds have the formulae:

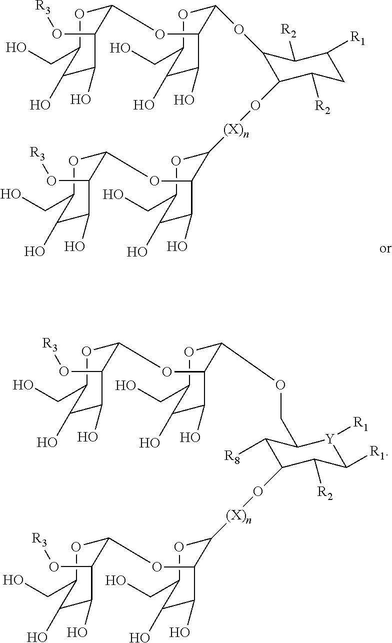

In these glycomimetics, there is no X where this n is 0, or X (as set forth above) is present where this n in 1.

Where n is 1 for (Z)ₙ, Z is present. Z is

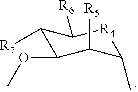

With X present, the glyeonrioiettcs of the eoropoun&s have the formulae:

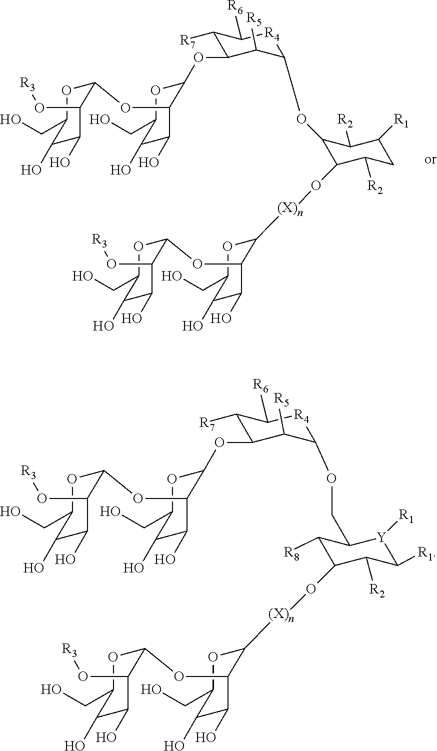

In these glycomiraclics, there is no X where this n is 0, or X (as set forth above) is present where this n is 1. Z possesses R₁, R₅, R₆and R₇. R₄ is a ring atom and may be either oxygen (O) or carbon (C). R₅ is H, C₁-C₈ alkanyl, aryl,

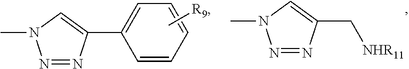

where R₉=F,
NH₂, C₁-C₈ alkanyl, C₂-C₈ alkenyl, aryl, COOH, or COOR₁₀, R₁₀=C₁-C₈ alkanyl, C₁-C₈ alkenyl, or aryl, R₁₁=C₁-C₈ alkanyl, C₂-C₈ alkenyl, or C(═O)R₁₂, R₁₂=C₁-C₈ alkanyl, C₂-C₈ alkenyL or aryl. R₆ is H, C₁-C₈ alkanyl, aryl, CH₂OH,

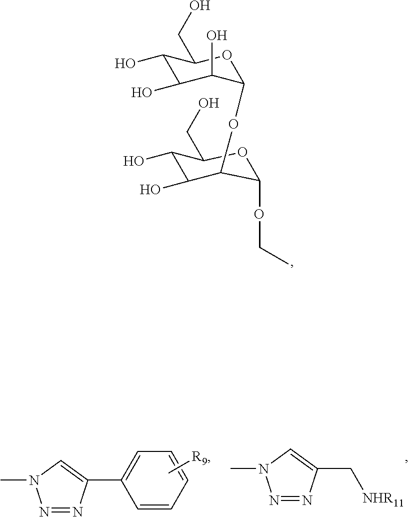

where R₉=NH₂, C₁-C₈ alkanyl, C₂-C₈ alkenyl, aryl, COOH, or COOR₁₀, R₁₀=C₁-C₈ alkanyl, C₂-C₈ alkenyl, or aryl, R₁₁-C₁-C₈ alkanyl, C₂-C₈ alkenyl, or C(═O)R₁₂, R₁₂-C_{1-C}₈ alkanyl, C₂-C₈ alkenyl, or aryl. R₇ is H or OH.

Other substiltients common to the above formulae are R₁, R₂ and R₃. R₁ is independently selected from H, C(═O)OCH₃ or L, with the proviso that both R₁ are not H or L (i.e., where there are two R₁ present on the same glycomimetic, the two R₁ are not both H and the two R₁ are not both L), and with the proviso where Y is oxygen that there is no R₁ at Y. R₂ is independently selected from H, C₁-C₈ alkanyl, C₂C₈ alkenyl, C₂-C₈ alkynyl, halogenated C₁-C₈ alkanyl, aryl or heteroeycle cither of which may be substituted with one or more of Me, OMe, halide, OH, or NHX where X=H, C₁-C₈ alkanyl, C₂-C₈ alkenyl C₂-C₈ alkynyl, halogenated C₁-C₈ alkanyl, aryl or heteroeycle either of which may be substituted with one or more of Me, OMe, haiide, or OH; —C(═O)OX where X is C₁-C₈ alkanyl, C₂-C₈ alkenyl, C₂-C₈ alkynyl, aryl or heterocyclc either of which may be substituted with one or more of Me, OMe, halide, or OH; —C(═O)NH(CH₂)ₙNH₂ where n=0-30, C(═O)NHX or CX₂OH, where X=C₁-C₈ alkanyl, C₂-C₈ alkenyl, C₂-C₈ alkynyl, halogenated C₁-C₈ alkanyl, aryl or heteroeycle cither of which may be substituted with one or more of Me, OMe, halide, or OH; OC(═O)X, OX, NHX, NH(═O)X, where X=H, C₁-C₈ alkanyl, C₂-C₈ alkenyl, C₂-C₈ alkynyl, halogenated C₁-C₈ alkanyl, aryl or heteroeycle either of which may be substituted with one or more of Me, OMe, halide, or OH;

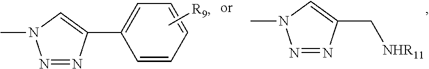

where R₉=F, NH₂, C₁-C₈ alkanyl, G₂-G₈ alkenyl, aryl, COOH, or COOR₁₀, R₁₀ =C₁-C₈ alkanyl, C₂-C₈ alkenyl, or aryl, R₁₁=C₁-C₈ alkanyl, C₂-C₈ alkenyl, or C(═O)R₁₂, R=C₁-C₈ alkanyl, C₂-C₈ alkenyl, or aryl. An example of Ra has the formula:

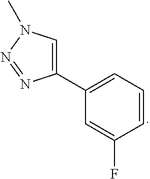

R₃ is H or mannose.

R₈ is specific to certain compound embodiments. R₈ is H, OH, CH₃, —(CH₂)ₘCH₃ where m is 1-20.

Where L is present, it is a linker. A linker may be biologically active or inactive. In one embodiment, the linker is biologically inactive. A linker may be (or may include) a spacer group, such as —(CH₂)ₚ— or —O(CH₂)ₚ—where p is generally about 1-20 (including any whole integer range therein). Other examples of spacer groups include a carbonyl or carbonyl containing group such as an amide. An embodiment of such spacer group is

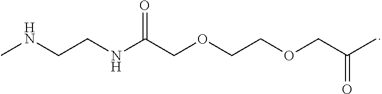 [SMILES: CNCCNC(=O)COCCOCC(C)=O]

Emodiments of linkers include the following:

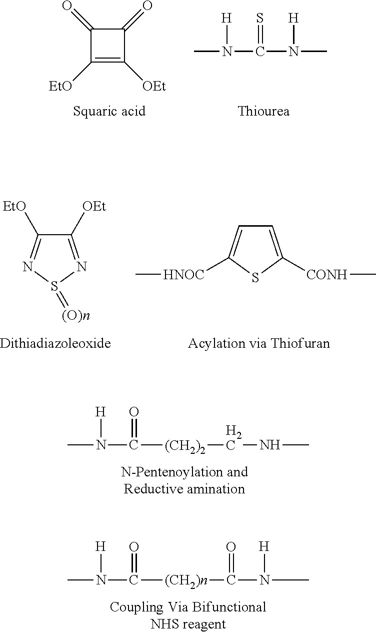

Other linkers, e.g, polyethylene glycols (PEG) or —C(═O)—NH_{(CH}₂)ₚ—C(═O)—NH₂ where p is as denned above, will be familiar to those in the art or in possession or the present disclosure.

In another embodiment, the linker is

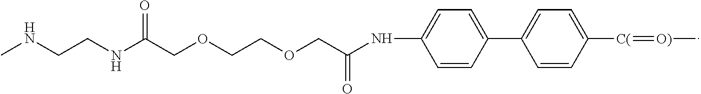

In another embodiment the linker is

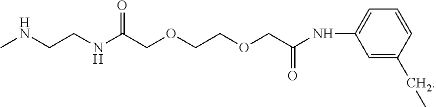 [SMILES: CCc1cccc(NC(=O)COCCOCC(=O)NCCNC)c1]

In another embodiment, the linker is —CH(═O)—NH—(CH₂)₂—NH—.

In another embodiment, the linker is —CH₂—NH—CH₂—.

In another embodiment, the linker is —C(═O)—NH—CH₂—.

As used herein, a “C₁-C₈ alkanyl” refers to an alkane substituent with one to eight carbon atoms and may be straight chain, branched or cyclic (cycloalkanyl). Examples are methyl (“Me”), ethyl, propyl, isopropyl, butyl and t-butyl. A “halogenated C₁-C₈ alkany” refers to a “C₁-C₈ alkanyl” possessing at least one halogen. Where there is more than one halogen present, the halogens present may be the same or different or both (if at least three present). A “C₂-C₈ alkenyl” refers to an aikene substituent with two to eight carbon atoms, at least one carbon carbon double bond, and may be straight chain, branched or cyclic (cycloalkenyl). Examples are similar to “C₁-C₈ alkanyl” examples except possessing at least one carbon carbon double bond. A “C₂-C₈ alkynyl” refers to an alkyne substituent with two to eight carbon atoms, at least one carbon-carbon triple bond, and may be straight chain, branched or cyclic (cycloalkynyl). Examples are similar to “C₁-C₈ alkaynly” examples except possessing at feast one carbon carbon triple bond. An “alkoxy” refers to an oxygen substituent possessing a “C₁-C₈ alkanyl,” “C₂-C₈ alkenyl” or “C₂-C₈ alkynyl.” This is —O-alkyl; for example methoxy, ethoxy, n-propoxy, i-propoxy, n-butoxy and the like; and alkenyi or alkynyl variations thereof (except for mcthoxy). It further refers to the group O-alkyl-W-alkyl where W is O or N; for example —O—(CH₂)_{n—W—(CH}₂)ₘ where n and m are independently 1-10. An “aryl” refers to an aromatic substituent with five to fourteen carbon atoms as ring atoms in one or multiple rings which may be separated by a bond or fused. As used herein, “heterocycle” includes aromatic and nonaromatic subsiituents. A “heterocycle” is a ringed substituent (one or multiple rings) that possesses at least one licteroutom (such as N, O or S) in place of a ring carbon. There are typically three to fourteen ring atoms. Examples of aryls and heterocycies include phenyl, naphthyl, pyridinyl, pyrimidinyl, triazolo, furanyl, oxazolyl, thiophenyl, quinolnyl and diphenyl.

At least two glycomimetics are joined to a “naphthalene” (i.e., unsubstituted naphthalene or substituted naphthalene), an “anthracene” (i.e., unsubstituted anthracene or substituted anthracene), a “phenalene” (i.e., unsubstituted phenalene or substituted phenalene), an “acenaphthylene” (i.e.. unsubstituted acenaphthylene or substituted acenaphthylene), or a “phenanthrene” (i.e., unsubstituted phenanthrene or substituted phenanthrene). Examples of substituents include C₁-C₈ alkanyl, halogenated C₁-C₈ alkanyl, alkoxy and halogens. Unsubstituted naphthalene

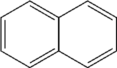

is which at least two linkers are attached, Unsubstituted anthracene is

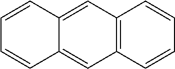 [SMILES: c1ccc2cc3ccccc3cc2c1]

to which at least two linkers are attached. Unsubstituted
phenlene is

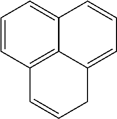 [SMILES: C1=Cc2cccc3cccc(c23)C1]

to which at least two linkers are attached.

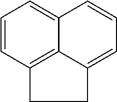 [SMILES: c1cc2c3c(cccc3c1)CC2]

Unsubstituted accnaphthylene is

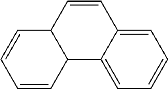 [SMILES: C1=CC2C=Cc3ccccc3C2C=C1]

to which at least two linkers are attached. Unsubstituted phenanthrene is to which at least two linkers are attached. Examples of naphthalene or phenalene include:

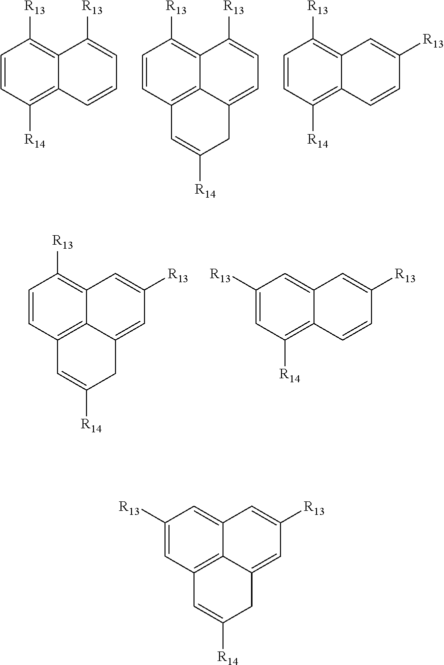

R₁₃ is NH or L. R₁₃ is used to attach to a glycomimetic. R₁₄ is H, CHO, L or L.A. L is a linker. L of R₁₄ is the same or different than L of R₁₃ . A is a vaccine carrier. Examples of a vaccine carrier include tetanus toxoid, keyhole limpet hemocyanin (KLH) or other protein carriers.

Compounds as described herein may be present within a pharmaceutical composition. A pharmaceutical composition comprises one or more compounds in combination with (i.e., not covalently bonded to) one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) or preservatives. Within yet other embodiments, compositions of the present invention may be formulated as a lyophilizate. Compositions of the present invention may be formulated for any appropriate manner of administration, including for example, topical, oral, nasal, intravenous, intracranial, intraperitoneal, subcutaneous, or intramuscular administration.

The compositions described herein may be administered as part of a sustained release formulation (i.e., a formulation such as a capsule or sponge that effects a slow release of compound following administration). Such formulations may generally be prepared using wolf known technology and administered by, for example, oral, rectal or subcutaneous implantation, or by implantation at the desired target site. Carriers for use within such formulations are biocompatible, and may also be biodegradable; preferably the formulation provides a relatively constant level of compound release. The amount of compound contained within a sustained release formulation depends upon the site of implantation, the rate and expected duration of release and the nature of the condition to be treated or prevented.

The above-described compounds including equivalents thereof are useful in methods of the present invention. In one embodiment, one or more of the compounds may be used in a method for inhibiting HIV infection in an individual. The individual may already have been exposed to HIV or may be at risk of such an exposure. Accordingly, the method may be for treating HIV infection or for preventing (prophylaxis) HIV infection. The method comprises administering in an amount effective to inhibit HIV infection a compound described herein. The compound may be with a pharmaceutically acceptable carrier or diluent.

The above-described compounds may be administered in a manner appropriate to the individual to be treated. Appropriate dosages and a suitable duration and frequency of administration may be determined by such factors as the condition of the patient, the type and severity of the patient's disease and the method of administration. In general, an appropriate dosage and treatment regimen provides the comnound(s) in an amount sufficient to provide therapeutic or prophylactic benefit. Within particularly preferred embodiments of the invention, a compound may be administered at a dosage ranging from 0.001 to 1000 mg/kg body weight (more typically 0.01 to 1000 mg/kg), on a regimen of single or multiple daily doses. Appropriate dosages may generally be determined using experimental models or clinical trials. In general, the use of the minimum dosage that is sufficient to provide effective therapy is preferred. Patients may generally be monitored for therapeutic effectiveness using assays suitable for the condition being treated, which will be familiar to those of ordinary skill in the art.

A compound or composition of the present invention can be used to develop therapeutic antibodies. Methods for producing therapeutic antibodies are well known in the art. The antibodies may be monoclonal antibodies. In an embodiment, the therapeutic antibodies may have been modified by domain swapping. Such methods are well known m the art. The therapeutic antibodies may be administered to an individual who already has been exposed to HIV or to an individual who may be at risk of such an exposure. Appropriate dosages and a suitable duration and frequency of administration may be determined by such factors as the condition of the patient, the type and severity of the patient's disease and ihe method of administration. In general, an appropriate dosage and treatment regimen provides the antibodies in an amount sufficient to provide therapeutic or prophylactic benefit. Appropriate dosages may generally be determined using experimental models or clinical trials. In general, the use of the minimum dosage that is sufficient to provide effective therapy is preferred. Patients may generally be monitored for therapeutic effectiveness using assays suitable for the condition being treated, which will be familiar to those of ordinary skill in the art.

A compound or composition of the present invention can be used as an inhibitor of HIV infection or in the manufacture of a medicament, for example for any of the uses recited herein. A medicament may include more than one compound or composition of the present invention. A medicament may include any compounds or compositions known at the time of ihe preparation of the medicament (e.g., one or more compounds useful in the prevention or treatment of HIV).

At least one (i.e., one or more) of the above described compounds may be administered in combination with at least one (i.e., one or more) anti-HIV agent. The compound may function independent of the agent, or may function in coordination with the agent, e.g., by enhancing effectiveness of the agent or vice versa. In addition, the administration may be in conjunction with one or more other therapies for reducing toxicities of therapy. For example, at least one (i.e., one or more) agent to counteract (at least in part) a side effect of therapy (e.g., anti-HIV therapy) may be administered. Agents (chemical or biological) that promote recovery are examples of such agents. At least one compound described herein may be administered before, after or simultaneous with administration of at least one agent or at least one agent to reduce a side effect of therapy. Where administration is simultaneous, the combination may be administered from a single container or two for more) separate containers.

The following Examples are offered by way of illustration and not by way of limitation.

### EXAMPLES

### Example 1

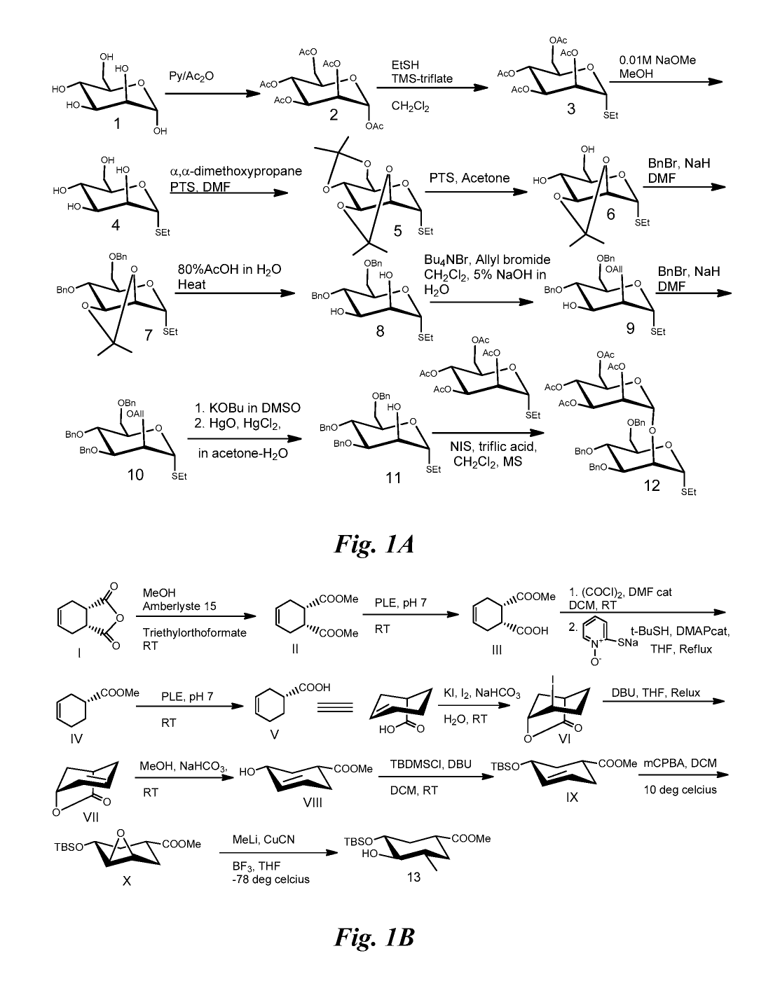

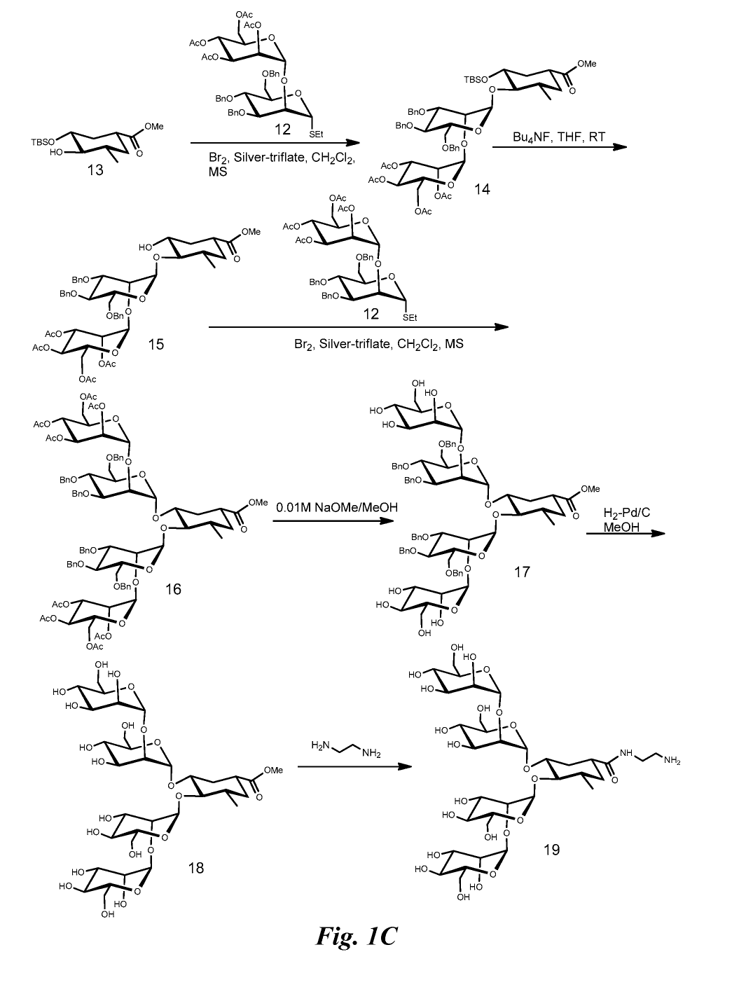

### Synthesis of A Representative Glycomimetic (Compound 19; FIG. 1)

### A. Synthesis of Compound 12 (FIG. 1A)

Synthesis of compound 3: Compound 3 (25 g) is synthesized as described in the literature (Carbohydr. Res. 193 (1989) 283-287).

Synthesis of compound 4: Compound 3 (20g) is stirred with 0.025 M NaOMc in MeOH (200 ml) for 4 h at room temperature. Neutralized with IR-120 (H+) resin, filtered and the liquid is evaporated to dryness to give compound 4 (12 g).

Synthesis of compound 3: Compound 4 (11.8 g) is co-evaporated with toluene (3×50 ml). The residue is dissolved in diy DMF (125 ml), and α,α-dimethoxy propane (60 ml) is added, followed by p-tohiene-sulfonic acid (0.125 g) with stirring at room temperature. Stirring is continued for 2 days at room temperature, neutralized with triethylamine (0.2 ml) and evaporated to dryness. The residue is dissolved in CH₂Cl₂ (70 ml) and washed with H₂O (3×50 ml). Organic layer is dried over Na₂SO₄filtered and concentrated to dryness. The residue is crystallized from hcxanes to give compound 5 (12 g).

Synthesis of compound 6: Compound 5 (11 g) is dissolved in acetone (160 ml), water (8 ml) and p-toluene-sulfonic acid monohydrate (0.8 g) is added with stirring at 40° C. The reaction mixture is stirred at 40° C. for 15 min. Triethylamine (1 ml) and NaHCO₃ (2 g) is added with stirring. The solution is then concentrated to dryness. Water (25 ml) is added and then extracted with hcxanes (2×75 ml). Aqueous layer is then extracted with CH₂Cl₂ (4×80 ml). Organic layer is dried (Na₂SO₄), filtered and evaporated to dryness. The residue ts crystallized from EtOAc-hexancs to give compound 6 (6 g),

Synthesis of compound 7: Compound 6 (5.5 g) is dissolved in DMF (40 ml) and cooled to 0° C. NaH (2.5 g, 60% dispersion in oil) is added with stirring. After 15 min, C₆H₅CH₂Br (7.7 ml) is added with stirring in the cold. Ice-bath is removed and the stirring is continued for 7 h at room temperature, followed by addition MeOH (5 ml) with Stirring at room temperature. The reaction mixture is concentrated to dryness, residue is dissolved in. CH₂Cl₂ (100 ml) and washed successively with brine, 1N HCI and brine. Organic layer is concentrated to dryness to give crude compound 7. It is used in the next step without further purification.

Synthesis of compound 8: Compound 7 (12 g crude) is dissolved in AcOH (20 ml), and water (5 ml) is added with stirring at 70° C. Stirring is continued for 1h at 70° C., solvent is evaporated off and the residue is crystallized from EiOAc-hexanes to compound 8 (5.2 g).

Synthesis of compound 9: Compound 8 (5 g) is dissolved in CH₂Cl₂ (200 ml). Allyl bromide (1.2 ml), Bu₄NBr (1.2 g) and 5% aqueous NaOH solution (20 ml) is added with Stirring. The reaction mixture is vigorously stirred at room temperature for 2 days. Organic layer is washed with H₂0 (4×150 ml), dried (Na₂SO₄) and concentrated to dryness. The residue is purified by column chromatography (silica gel) to give compound 9 (4.5 g).

Synthesis of compound 10: Compound 9 (4 g) is dissolved in DMF (30 ml) and cooled to 0° C. NaH (0.64 g, 60% dispersion in oil) is added with stirring. After 15 min, C₆H₅CH₂Br (2.8 ml) is added vvith stirring in the cold. Ice-bath is removed and the stirring is continued for 7h at room temperature followed by addition MeOH (5 ml) with stirring at room temperature. The reaction mixture is concentrated to dryness, residue is dissolved in CH₂Cl₂ (100 ml) and washed successively with brine, 1N HCl and brine. Organic layer is concentrated to dryness and purified by column chromatography (silica gel) to give compound 10 (4.2 g).

Synthesis of compound 11: To a solution of compound 10 (4 g) in dry DMSO (20 ml) is added potassium tert-butoxide (0.5 g) and the reaction mixture is stirred, at 100° C. for 2h under dry nitrogen. The reaction mixture is cooled down to room temperature and H₂) (40 ml) is added with stirring. The reaction mixture is extracted with CH₂Cl₂ (4×50 ml). The organic layer is washed with H₂O (3×40 ml) and concentrated to dryness. The residue is dissolved in CH₃COCH₃—H₂O (10:1, 33 ml), yellow mercuric oxide (2 g) is added with stirring and then a solution of HgCl₂ (2 g) in CH₃COCH₃—H₂O (10:1, 20 ml) is added dropwise with stirring at room temperature. After 30 min, the reaction mixture is filtered through Celite and concentrated to dryness. Diethylether (100 ml) is added and the solution is washed vv ith a saturated solution of KI (1×50 ml) and water (2×50 ml). The organic layer is concentrated to dryness and purified by column chromatography (silica gel) to give compound 11 (2.2 g).

Synthesis of compound 12: A mixture of compound 11 (2 g), compound 3 (2.4 g), and activated powdered molecular sieves (4Å, 2 g) in dry CH₂Cl₂ (50 ml) is stirred at room temperature for 1h under argon. The mixture is cooled to 0-5° C. (ice-bath) and NIS (2.2 g) is added while stirring in the cold. A 0.15 M solution of trillic acid in CH₂Cl₂(10 ml) is added dropwise over 30 min with stirring in the cold. After 1h, the reaction mixture is filtered through Celite and washed successively with cold 5% aqueous solution of Na₂S₂O₃, saturated solution of NaHCO₃, and H₂O. The organic layer is concentrated to dryness and purified by column chromatography (silica gel) to give compound 12 (2.7 g).

### B. Synthesis of Compound 13 (FIG. 1B)

Synthesis of compound II: Commercially available cis-1,2,3,4-tetrahydrophthalic anhydride (I, 50 g) is added to a suspension of amberlyste 35 (50 g, dried under vacuum) in methanol (1L) with stirring. Triethylorthoformate (100 ml) is added immediately while stirring. The reaction mixture is then vigorously stirred for 5 days at room temperature and additional triclhylorthoformate is added. Stirring is continued for an additional 4 days, filtered over celite and washed with methanol. The solvent is removed in vacuum and the residue is dissolved in CH₂Cl₂ (200 ml). The solution is washed with cold saturated solution of NaHCO₃ (200 ml) and cold brine (200 ml). The organic layer is dried (Na₂SO₄), filtered and concentrated to dryness to afford compound II (55 g).

Synthesis of compound III: To a suspension of compound II (10 g) in phosphate buffer (400 ml, pH 7) is added PLE (40 mg, 1080 unit). The pH of the mixture is maintained at 7 by continuous drop wise addition of 1M NaOH solution via syringe pump. The reaction is stirred at 20° C. until 1 equivalent of NaOH (50 ml) is used. The reaction mixture is transferred to a seperatory funnel and EtOAc (400 ml) is added. The layers are separated and the organic layer extracted with phosphate buffer (2×250 ml, pH 7). The combined aqueous layers are acidified (pH 2) with aqueous HCl (1M) and extracted with EtOAc (3×400 ml). The combined organic layers are dried (Na₂SO₄). filtered and concentrated to dryness to afford compound III (7.8 g).

Synthesis of compound IV: To a solution of compound III (2 g) in dry CH₂Cl₂ (35 ml) is added (COCl); (1.4 ml) and DMF (0.025 ml) and stirred for 3h at RT. The solution is evaporated to dryness (rotavapor is purged with argon). The residue is dissolved in dry THF (40 ml) and added dropwise over a period of 20 min to a boiling suspension of 2-mercaptopyridine-1-oxid sodium salt (2 g), t-BuSH (6 ml), and 4-DMAP (52 mg) in dry THF (100 ml). The solution is stirred under reflux for 3 h. The reaction mixture is cooled down to RT and transferred into a seperatory funnel with EtOAc (100 ml) and washed with H₂O (100 ml). The aqueous layer is extracted with EtOAc (2×200 ml). The combined organic layers are dried (Na₂S₄), filtered and concentrated to dryness. The crude product is purified by column chromatography (silica) to afford compound IV as yellowish oil (1.1 g).

Synthesis of compound V: To a suspension of compound IV (4 g) in phosphate buffer (400 ml, pH 7) is added PLE (42 mg) with stirring. The pH is kept at 7 by adding NaOH solution (1 M) via syringe pump. The reaction mixture is stirred at RT until 1 equivalent ofNaOH is used. The reaction mixture is transferred to a seperatory funnel and washed with EtOAc (2×250 ml). The layers are separated and the organic layers extracted with phosphate buffer (2×250 ml, pH 7). The combined aqueous layers are acidified to pH 2 with aqueous HCl solution and extracted with EtOAc (3×300 ml). The combined organic layers are dried (Na₂SO₄), filtered and evaporated to dryness. The crude product is filtered through a short plug of silica to afford compound V (3 g).

Synthesis of compound VI: Compound V (4 g) is suspended in water (90 ml) and cooled down to 0° C. NaHCO3 (8 g) is added followed by a solution of Kl (32 g) and I₂ (8 g) in water (75 ml). The reaction mixture is stirred at RT for 24 h and then extracted with CH₂Cl₂ (3×30 ml). The combined organic layers are washed with a saturated solution of Na₂S₂O₃ in water (125 ml). The aqueous layer is extracted with CH₂Cl₂ (2×30 ml). The combined organic layers are protected from light, dried (Na₂SO₄), filtered, and concentrated to dryness and quickly under high vacuum to afford iodolaetone VI as an off-white solid (7.5 g).

Synthesis of compound VII: Compound VI (7 g) is dissolved in dry THF (170 ml) and DBU (7 ml) is added. The reaction mixture is refluxed for 20 h and then cooled downed to RT. Diethyl ether (100 ml) is added and transferred into a separatory funnel and extracted with aqueous solution of HCI (200 ml, 0.5 M). The aqueous layers are extracted with Et₂O (3×100 ml). The combined organic layers are washed with brine (200 ml), dried (Na₂SO₄), filtered, and concentrated to dryness. The crude product is purified by column chromatography (silica gel) to afford compound VII (3.7 g).

Synthesis of compound VIII: NaHCO₃ (2.2 g) is dried under vacuum and then dry MeOH (132 ml) is added with stirring followed by compound VII (3 g). The reaction mixture is then stirred at RT under argon for 12 h. The solvent is evaporated off and the residue transferred into a sepcratory funnel with CH₂Cl₂ (35 ml), extracted with water (40 ml) and with brine (40 ml). The aqueous layer is extracted with CH₂Cl₂ (2×35 ml). The combined organic layers are dried (Na₂SO₄), filtered, and concentrated to dryness to give compound VIII (5 g).

Synthesis of compound IX: To a solution of compound VIII (4 g) in dry CH₂Cl₂ (80 ml) is added tert-butyidimethylsilyl chloride (7.2 ml) in small portions, followed by DBU (9.5 ml). The reaction mixture is stirred for 12 h and then quenched with MeOH (12 ml). The reaction mixture is transferred into a sepcratory funnel with CH₂Cl₂ (60 ml), washed with cold saturated solution of NaHCO₃ (50 ml) and cold brine (50 ml). The aqueous layers are extracted with CH₂Cl₂ (2×50 ml). The combined organic layers are dried (Na₂SO₄), filtered and concentrated to dryness. The residue is purified by column chromatography (silica) to give compound IX (6 g).

Synthesis of compound X: To a cold (10° C.) solution of compound IX (5 g) in CH₂Cl₂ (125 ml) is added m-CPBA (8 g) with stirring and continued to stir for 15 h at 10° C. The temperature is raised to RT over a period of 2 h and the mixture diluted with CH₂Cl₂ (400 ml). The mixture is transferred into a sepcratory runnel, washed with a cold saturated solution ofNa₂S₂O₃ solution in water (2×400 ml). The organic layer is successively washed with cold saturated solution NaHCO₃ (400 ml) and cold brine (100 ml). The aqueous layers are extracted with CH₂Cl₂ (2×400 ml). The combined organic layers are dried (Na₂SO₄), filtered, and concentrated to dryness. The crude product is purified by column chromatography (silica) to give compound X (4 g).

Synthesis of compound 13: CuCN (1.5 g) is dried in high vacuum at 150° C. for 30 min, suspended in dry THF (25 ml) and cooled down to −78° C. MeLi (1.6 M in Et₂O, 22.5 ml) is added slowly via syringe and the temperature raised to −10° C. over a period of 30 min. The mixture is again cooled down to −78° C. followed by the addition of BF₃ etherate (1.4 ml) in THF (5 ml). After stirring for 20 min. compound X (1 g) in THF (25 ml) is added and stirring continued for 5 h at −7° C. The excess of MeLi is quenched with mixture of MeOH (10 ml) and Et₃N (10 ml). The mixture is diluted with Et₂O (250 ml) and transferred into a sepcratory funnel and extracted with an aqueous 25% NH/ₑ/satd. NH₄Cl (1:9) solution. The organic layer is successively washed with brine (150 ml), 5% AcOH (150 ml), saturated solution of NaHCO₃(150 ml), and brine (150 ml). The aqueous layers are extracted with Et₂O (2×250 ml). The combined organic layers are dried (Na₂SO₄), filtered, and concentrated to dryness. The crude product is purified by column chromatography (silica) to give compound 13 (800 mg).

### C. Synthesis of Compound 19 (FIG. 1C)

Synthesis of compound 14: To a solution of compound 13 (1 g) in CH₂Cl₂ (25 ml) is added powdered molecular sieves (4Å, 1 g) and compound 12 (2.8 g). The reaction mixture is allowed to stir at room temperature for 2 h at under argon. Silver trifluoromethanesulfonate (1.5 g) is added, and stirring is continued for 15 min. then Br₂ (0.1 ml) is added and the reaction mixture is stirred for a further 2 h under argon. Triethylamine (0.5 ml) is added and the reaction mixture is filtered through a bed of Celite. CH₂Cl₂ (100 ml) is added and the organic layer is successively washed with 5% Na₂S₂O₃ (50 ml), saturated solution of NaHCO₃ (50 ml), and H₂O (50 ml). Organic layer is concentrated to dryness and the residue is purified by column chromatography (silica gel) to give compound 14 (2 g).

Synthesis of compound 15: To a solution of compound 14 (1.8 g) in THF (15 ml) is added a solution of ictrabutylammomum fluoride (9.6 ml) and the reaction mixture is stirred at room temperature tor 24 h. Solvent is evaporated off and the residue is purified by column chromatography (silica gel) to give compound 15 (1.5 g).

Synthesis of compound 16: To a solution of compound 15 (1.4 g) in CH₂Cl₂ (15 ml) is added powdered molecular sieves (4Å, 0.5 g) and compound 12 (1.4 g). The reaction mixture is stirred at room temperature for 2 h under argon. Silver tirifluoromcethanesulfonate (0.8 g) is added, and stirring is continued for 15 min, then Br₂ (0.05 ml) is added and the reaction mixture is stirred for a further 2 h under argon. Triethylamine (0.25 ml) is added and the reaction mixture is filtered through a bed of Celiie. CH₂Cl₂ (50 ml) is added and the organic layer is successively washed with 5% Na₂S₂O₃ (25 ml), saturated solution of NaHCO₃ (25 ml), and H₂O (25 ml). Organic layer is concentrated to dryness and the residue is purified by column chromatography (silica gel) to give compound 16 (1.2 g).

Synthesis of compound 17: Compound 16 (1 g) is stirred with 0.025 M NaOMe in MeOH (10 ml) for 4 h at room temperature. Neutralized with IR-120 (H+) resin, filtered and the liquid is evaporated to dryness to give compound 17 (0.5 g).

Synthesis of compound 18: Compound 17 (0.45 g) is dissolved in McOH (5 ml) and 10% Pd-C (0.25 g) is added. The reaction mixture is shaken under hydrogen for 24 h at room temperature. The reaction mixture is filtered through Celite and the filtrate is evaporated to dryness to give compound 18 (0.25 g). Synthesis of compound 19: Compound 18 (0.2 g) is treated with ethylenediaminc (2 ml) at room temperature overnight, solvent is evaporated off and the residue is purified by sephadex G-10 column to give compound 19 (0.15 g).

### Example 2

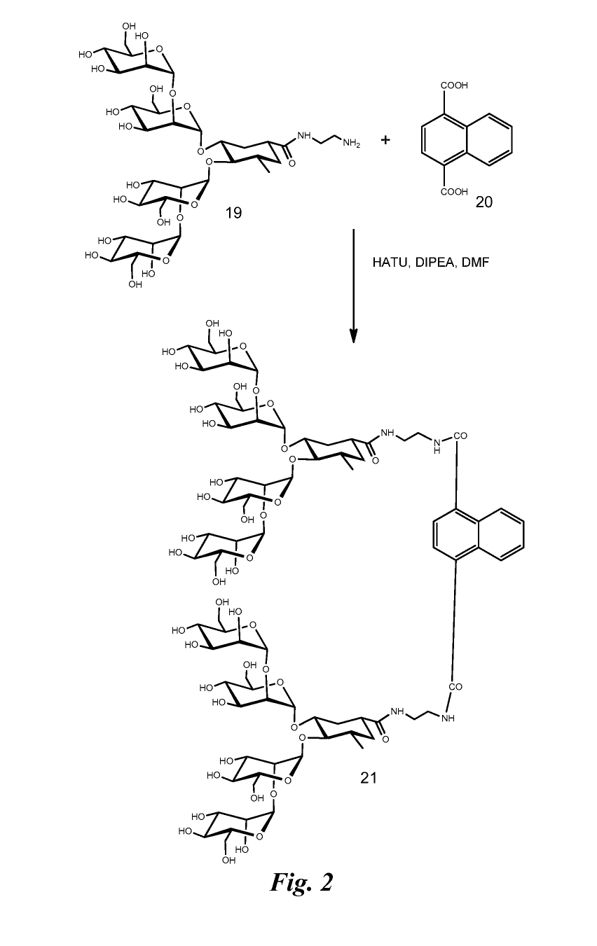

### Synthesis of a Representative Compound (Compound 21; FIG. 2)

Synthesis of compound 21: To a solution of commercially available compound 20 (12 mg, Aldrich chemical company, St. Louis, Mo.) in DMF (0.25 ml) is added N,N-Diisopropylethylamine (0.022 ml) and HATU (0.060 g) and stirred for 3 min at room temperature. To this reaction mixture is added compound 19 (0.1 g) from Example 1, and the reaction mixture is stirred for 30 min at room temperature. The reaction mixture is concentrated to dryness and the residue is first passed through a sep-pak C18 cartridges and then purified by reverse-phase hplc to give compound 21 (0.07 g).

### EXAMPLE 3

### DC-Sign Assay

1. Coat probind 96-well microliter plate: DC-Sign (ECD)
a) Add DC-Sign (R&D Systems. Minneapolis, Minn.) 100 μl /well of 3 μg/ml to columns 1-11
b) Buffer only [Tris-Ca⁺²] to column 12

2. Incubate: 2 hours at 37° C. covered

3. Block: with BSA
a) Prepare 1% BSA
b) Add 100 μ/well of 1% BSA in (Tris-Ca⁺²)

4. Incubate: 2 hours at room temp, covered

5. Prepare samples in separate round bottom plate: 1
a) Prepare compounds in (Tris-Ca⁺²) with 10% DMSO
b) Add 120 μl of compounds to column 1, then 2X dilutions to columns 2-9
c) Buffer only [1% BSA in (Tris-Ca⁺²)] to columns 10 & 12 60 μl, and to column 11 120 μl

6. Add Le^{a}-PAA-biotin/SA-HRP to round bottom plate
a) Le^{a}-PAA (GlycoTech Corp., Rockville, Md.) is pre-incubated (24 hours) with sStreptavidin-labeled horseradish peroxidase (SA-HRP) to form Le^{a}-PAA/SA-HRP polymer.
b) Add 60 μl/well of 0.5 μg/ml Le^{a}-PAA-biotin/SA-HRP polymer to columns 1-10 & 12.

7. Wash probind plate: 4 times with Tris-Ca⁺²

8. Transfer samples: 100 μl/well from round bottom plale to probind plate

9. Incubate: 2 hours at room temp. covered and rotating

10. Wash probind plate: 4 times with Tris-Ca⁺²

11. Add TMB (3,3′,5,5′-tetramethyl benzidine):H₂O₂: 100 μl/well

12. Incubate: 3 min at room temp.

13. Add H₃PO₄: 100 μl/well of 1M solution to H₃PO₄ solution to stop reaction

14. Plate reader: read at 450 nm

### Example 4

### Immunoassay to Determine the Binding of Glycomimetic Antigen for Antibody 2G12

1. Coal wells of a 96 microtiter plate with gp120 (Advanced Bioscience Labs, Kensington, Md. overnight in phosphate buffered saline (PBS) pH 7.4 at 4° C.

2. Wash plate with PBS and block wells with 1% BSA in PBS pH 7.4 for 2 hours at room temperature.

3. Acid 50 ul of glycomimetic antigen in 1% BSA, PBS pH 7.4 serially diluted from well 1 to 11, with well 12 containing buffer but no antigen.

4. Add 50 ul of antibody 2G12 (Polymun Scientific, Vienna, Austria) diluted in 1% BSA. PBS pH 7.4 to each well.

5. Incubate rotating at room temperature for 2 hours.

6. Wash plate with PBS and add secondary antibody (Pierce Chemical Co., Roekford, Ill.) conjugated with horseradish peroxidase (2 ug/ml) in 1% BSA, PBS pH 7.4.

7. Incubate rotating at room temperature for 1 hour.

8. Wash and add TMB (3,3′5,5′-letramethyl benzidine) reagent (100 ul/well) to each well. Wait 10 minutes. Stop reaction by adding 100 ul of 1 M phosphoric acid to each well, and read optical density at wavelength 450 nm.

The various embodiments described above can be combined to provide further embodiments. All of the U.S. patents. U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is clamed is:

1-20. (canceled)

21. A compound chosen from glycomimetics of the following formulas: 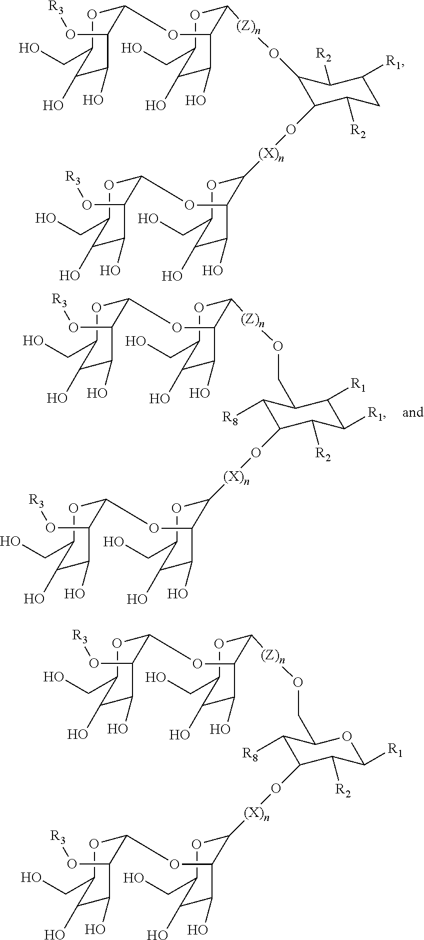
wherein
n, which may be identical or different, are each independently chosen from 0 and 1;
Z, which may be identical or different, are each independently chosen from 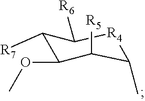
X is 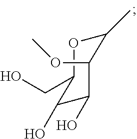
R₁, which may be identical or different, are each independently chosen from H, —C(═O)OCH;, and —L;
R₂, which may be identical or different, are each independently chosen from H, C₁-C₈ alkanyl, C₂-C₈ alkenyl, C₂-C₈ alkynyl, halogcnated C₁-C₈ alkanyl, aryl, heterocyclc, —C(═O)OX¹, —C(═O)NH(CH₂)_{y}NH₂, —C(═O)NHX², —C(X²)₂OH, —OC(═O)H, —OC(═O)X², —OH, —OX², —NH₂, —NHX₂, 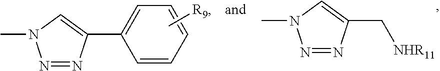
wherein y, which may be identical or different, are each independently chosen from integers ranging from 0 to 30, wherein X¹, which may be identical or different, are each independently chosen from C₁-C₈ alkanyl, C₂-C₈ alkenyl, C₂-C₈ alkynyl, aryl, and heterocycle, wherein X₂, which may be identical or different, are each independently chosen from C₁-C₈ alkanyl, C₂-C₈ alkenyl, C₂-C₈ alkynyl, halogcnated C₁-C₈ alkanyl, aryl, and heterocycle;
R₃, which may be identical or different, are each independently chosen from H and mannose;
R₄, which may be identical or different, are each independently chosen from O and C;
R₅, which may be identical or different, are each independently chosen from H, C₁-C₈ alkanyl, aryl, 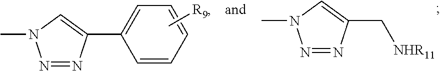
R₆, which may be identical or different, arc each independently chosen from H, C₁-C₈ alkanyl, aryl, —CH₂OH, 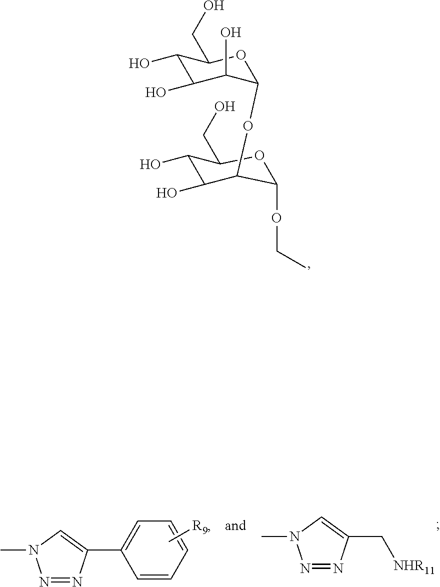
R₇, which may be identical or different, are each independently chosen from H and OH;
R₈ is chosen from H, OH, CH₃, and —(CH₂)ₘCH₃, wherein m is chosen from integers ranging from 1 to 20;
R₉, which may be identical or different, are each independently chosen from F, NH₂, C₁-C₈ alkanyl, C₂-C₈ alkenyl, aryl, COOH, and COOR₁₀;
R₁₀, which may be identical or different, are each independently chosen from C₁-C₈ alknyl, C₂2-C₈ alkenyl, and aryl;
R₁₁, which may be identical or different, are each independently chosen from C₁-C₈ alkanyl, C₂-C₈ alkenyl, and C(═O)R₁₂;
R₁₂, which may be identical or different, are each independently chosen from C₁-C₈ alkanyl, C₂-C₈ alkenyl, and aryl; and
L, which may be identical or different, are each independently chosen from linker groups.

22. The compound according to claim 21, wherein n is 0 for cither (Z)ₙ or (X)ₙ or both.

23. The compound according to claim 22, wherein the compound is chosen from glycomimetics of the following formula:

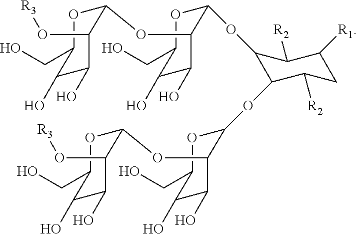

24. The compound according lo claim 22, wherein the compound is chosen from glycomimetics of the following formulas:

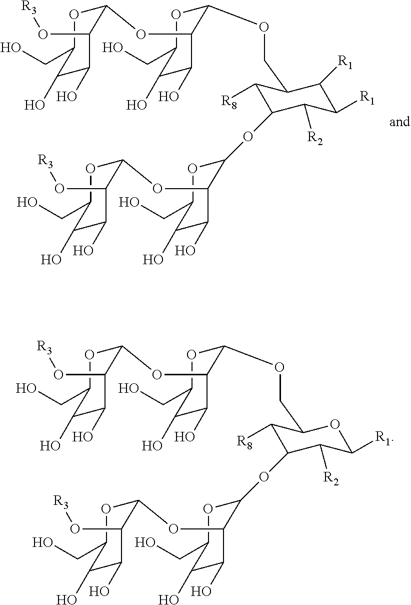

25. The compound according to claim 21, wherein n is 1 for either (Z)ₙ or (X)ₙ or both.

26. The compound according to claim 25, wherein the compound is chosen from glycomimetics of the following formulas:

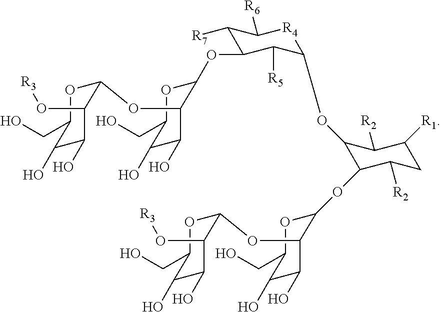

27. The compout according to claim 26, wherein R₆ is

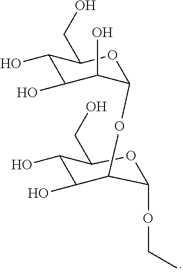 [SMILES: CCOC1OC(CO)C(O)C(O)C1OC1OC(CO)C(O)C(O)C1O]

28. The compound according to claim 25 , wherein is glycomimetics of the following formulas:

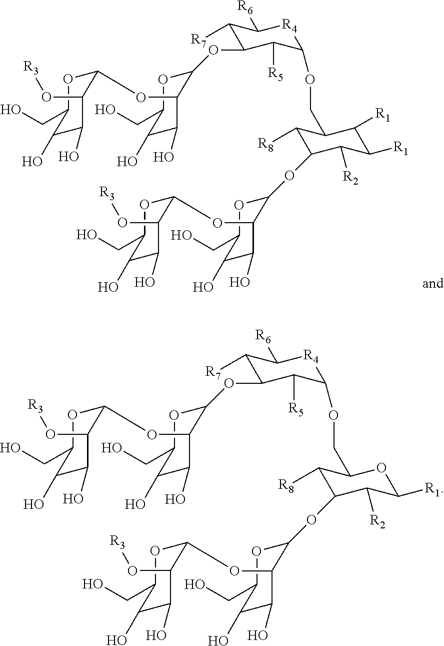

29. The compound according to claim 28 , wherein at least one R₆ is

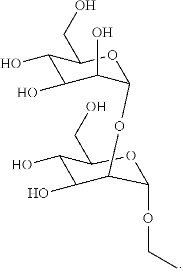 [SMILES: CCOC1OC(CO)C(O)C(O)C1OC1OC(CO)C(O)C(O)C1O]

30. The compound according to claim 21, wherein at least one R₄ is O.

31. The compound according to claim 21, wherein at least one R₄ is C.

32. The compound accroding to claim 21, wherein at least one R₂ is aryl.

33. The compound according to claim 21, wherein at least one R₂ has the formula:

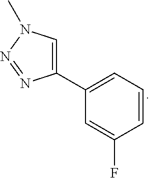

34. A compound comprising at least two glycomimetic groups independently chosen from glycomimetic groups having the following formulas: 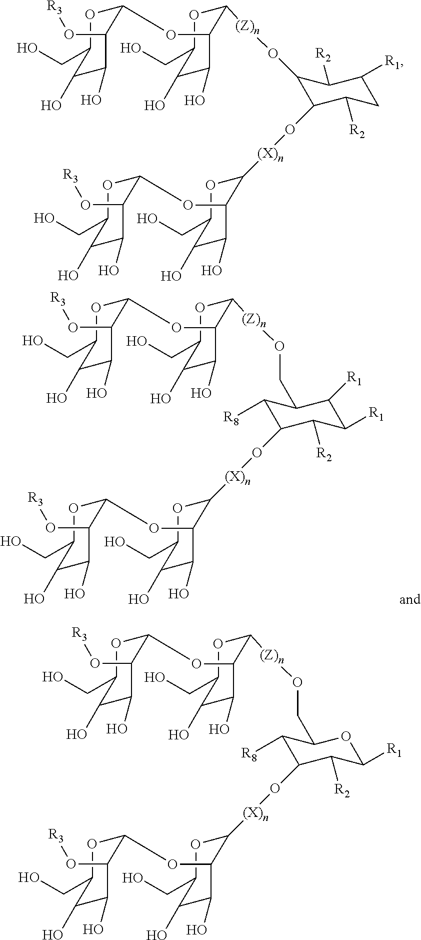
wherein
n, which may be identical or different, are each independently chosen from 0 and 1;
Z, which may be identical or different, are each independently chosen from 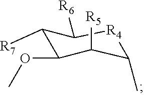
X is 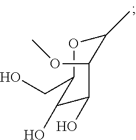
R₁, which may be identical or different, are each independently chosen from H, —C(═O)OCH₃, and —L, with the proviso that exactly one R₁ on each glycomimetic group is —L;
R₂, which may be identical or different, are each independently chosen from H, C₁-C₈ alkanyl, C₂-C₈ alkenyl, C₂-C₈ alkynyl, halogenated C₁-C₈ alkanyl, aryl, heterocycle, —C(═O)OX¹, —C(═O)NH(CH₂)_{y}NH₂, —C(═O)NHX², —C(X²)₂OH, —OC(═O)H, —OC(═O)X², —OH, —OX², —NH₂, —NHX², 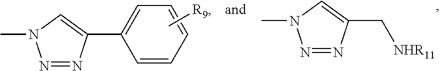
wherein y, which may be identical or different, are each independently chosen from integers ranging from 0 to 30, wherein X¹, which may be identical or different, are each independently chose from C₁-C₈ alkanyl, C₂-C₈ alkenyl, C₂-C₈ alkynyl, aryl, and heterocycle, whrein X², which may be identical or different, are each independently chosen from C₁-C₈ alkanyl, C₂-C₈ alkeny, C₂-C₈ alkynyl, halogenated C₁-C₈ alkanyl, aryl, and heterocycle;
R₃, which may be identical or different, are each idependently chosen from H and mannose;
R₄, which may be identical or different, are each independently chosen from O and C;
R₅, which may be identical or different, are each independently chosen from H, C₁-C₈ alkanyl, aryl, 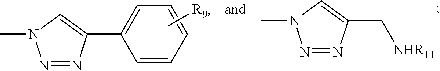
R₆, which may be identical or different, are each independently chosen from H, C₁-C₈ alkanyl, aryl, —CH₂OH, 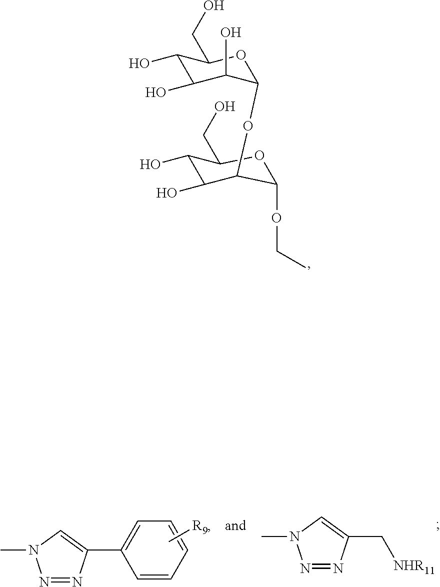
R₇, which may be identical or different, are each independently chosen from H and OH;
R₈, which may be identical or different, are each independently chosen H, OH, —CH₃, and —(CH₂)ₘCH₃, wherein m, which may be identical or different, are each independently chosen from integers ranging from 1 to 20;
R₉, which may be identical or different, are each independently chosen from F, NH₂, C₁-C₈ alkanyl, C₂-C₈ alkenyl, aryl, COOH, and COOR₁₀;
R₁₀, which may be identical or different, are each independently chosen from C₁-C₈ alkanyl, C₂-C₈ alkenyl, and aryl;
R₁₁, which may be identical or different, are each independently chosen from C₁-C₈ alkanyl, C₂-C₈ alkenyl, and C(═O)R₁₂;
R₁₂, which may be identical or different, are each independently chosen from C₁-C₈ alkanyl, C₂₋C8 alkenyl, and aryl; and
L, which may be identical or different, are each independently chosen from linker groups.

35. A composition comprising at least one compound according to claim 34 and optionally at least one pharmaceutically acceptable carrier or diluent.

36. A method for inhibiting HIV infection comprising administering to an individual in need thereof an effective amount of at least one compound according to claim 34.

37. The method according to claim 36, further comprising administering at least one pharmaceutically acceptable carrier or diluent.
